(19) United States
(12) Patent Application Publication
(10) Pub. No.: US 2004/0181156 A1
(43) Pub. Date: Sep. 16, 2004

(54) Inflatable products and methods of their formation and use

(76) Inventors: Howard A. Kingsford, Amherst, NH (US); Holly Isabelle, Deerfield, NH (US); George A. Provost, Litchfield, NH (US); William H. Shepard, Amherst, NH (US)

Correspondence Address: FISH & RICHARDSON PC, 225 FRANKLIN ST, BOSTON, MA, 02110, US

(21) Appl. No.: 10/738,860

(22) Filed: Dec. 16, 2003

Related U.S. Application Data
(60) Provisional application No. 60/434,085, filed on Dec. 16, 2002.
(60) Provisional application No. 60/494,653, filed on Aug. 12, 2003.
(60) Provisional application No. 60/494,659, filed on Aug. 12, 2003.

(51) Int. Cl. A61B 5/02; B63C 9/08; B61D 45/00

(52) U.S. Cl. 600/490; 600/499; 441/122; 410/119; 264/241

(57) ABSTRACT

An inflatable product includes overlapped areas of flexible material forming an inflatable pouch that is bounded by bonded regions of the flexible material and that includes a limited opening for inflating the pouch. The flexible material includes multiple fastening regions including a first fastening region in which an array of fastener elements with stems are arranged, the stems are integrally molded with and extending from a common resin substrate, and a second fastening region of fibrous loops releasably engageable with the fastener elements.

### CROSS-REFERENCE TO RELATED APPLICATIONS

This application claims priority to U.S. Provisional Application Serial No. 60/434,085, filed on Dec. 16, 2002, to U.S Provisional Application Serial No. 60/494,653, filed on Aug. 12, 2003 and to U.S. Provisional Application Serial No. 60/494,659, filed on Aug. 12, 2003.

### TECHNICAL FIELD

This invention relates to inflatable pouches and other products with reclosable fasteners.

### BACKGROUND

Self-securing, inflatable pouches have ordinarily involved relatively expensive designs and manufacturing techniques. Efforts to make pouches of lower cost can limit their functionality or convenience.

### SUMMARY

The invention features, in several of its aspects, an inflatable product having an inflatable pouch formed between overlapped areas of flexible material and multiple regions of engageable fastener elements. Among other applications, this product can be used for medical applications, such as a relatively low-cost, single-use blood pressure cuff or splint.

According to one aspect of the invention, an inflatable product includes overlapped areas of flexible material defining therebetween an inflatable pouch that is bounded by bonded regions of the flexible material and includes a limited opening for inflating the pouch. The flexible material includes multiple fastening regions including a first fastening region in which an array of fastener elements with stems are arranged, the stems integrally molded with and extending from a common resin substrate. The flexible material also includes a second fastening region of fibrous loops releasably engageable with the fastener elements.

In some embodiments, the inflatable product includes a valve to inhibit egress of fluid from the inflatable pouch through the limited opening, once inflated. The valve can include a passage that is collapsible in response to internal pouch pressure. Where the valve includes a collapsible passage, the valve can be formed of two sheets of flexible material sealed together in face-to-face relation along either side of the passage. In some cases, the valve includes a removable plug that can seal the opening.

In some cases, the resin substrate from which the fastener element stems extend comprises a region of the flexible material. In some embodiments, the second fastening region and/or the array of fastener element stems overlaps the pouch. In certain embodiments, the second fastening region covers substantially all of a front side of the product. In some cases, the resin substrate is bonded to and extends from an edge of the flexible material.

In some embodiments, the pouch includes more than one compartment, such as multiple hydraulically connected compartments defined between extensions of the bonding regions.

In certain applications, the opening is defined within a fitment secured to the flexible material. In these cases, the fitment can be configured to mate with a flexible hose for hydraulic communication with the pouch.

In certain embodiments, the inflatable product includes a compartment defined between adjacent areas of the flexible material and bounded by bonded regions of the flexible film material, the compartment defining an opening for receiving an object, such as a thermal pad. In cases where the inflatable product includes a compartment, the first and second fastening regions can be positioned at the opening of the compartment to form a releasable closure for releasably closing the opening of the compartment. In certain cases, the compartment and the inflatable pouch share a wall of flexible material.

In some embodiments, the flexible material forms a strap carrying at least one of the first and second fastening regions, the strap being of sufficient length to wrap about an object and engage the first and second fastening regions to secure the pouch to an object.

In many embodiments, the flexible material comprises film, such as a film having a thickness of between about 0.0005 and 0.008 inch (between about 0.00127 and 0.02032 cm), preferably between about 0.001 and 0.005 inch (between about 0.00254 and 0.0127 cm), such as about 0.004 inch (about 0.01016 cm). In some embodiments, the flexible material is polyethylene.

The fibrous loops can be formed from a loop material selected from a group consisting of a non-woven material, a woven material and a knit material. In some cases, the loop material is bonded to the flexible material. In certain applications, the loop material is a dimensionally stable loop material.

In some embodiments, the fastener elements include loop-engageable heads extending laterally from the stems in discrete directions. In other embodiments, the fastener elements include loop-engageable heads extending laterally from the stems in multiple directions.

In some applications, the fastening regions are formed to enable removal of the inflatable product by application of a peeling force of less than about 0.2 pounds of force per transverse inch, such as less than about 0.1 pounds of force per transverse inch of engaged width of the engaged fastener elements.

In certain cases, the inflatable product has an overall weight of less than about 150 grams per square meter of overall area of one broad side of the product, in an uninflated state.

In some embodiments, the inflatable product is in the form of a blood pressure cuff and can include a fitment for attachment to a flexible hose, the fitment providing access to the pouch. In some cases, the blood pressure cuff includes a second fitment. In some embodiments, the fitment includes more than one passageway.

In certain embodiments, the inflatable product is in the form of a reclosable bag, the flexible material forming a compartment for containing an object.

In some cases, the inflatable product is in the form of a splint for confining a body part of a patient. In these cases, the splint can include a hole therethrough, shaped to receive a part of a human body.

In certain cases, the inflatable product is in the form of a floatation device and formed to be strapped about a human body. In these cases, the inflatable product can be a life jacket and capable of inflating to an amount capable of keeping a person afloat in water.

According to another aspect of the invention, an inflatable blood pressure cuff includes a compartment defined between overlapped areas of flexible film material and bounded by bonded regions of flexible film material, the compartment having an opening sized to receive an inflatable pad. The blood pressure cuff also includes an inflatable pad positioned within the compartment with the compartment formed to allow the inflatable pad to expand while positioned within the compartment. The flexible film material of the blood pressure cuff includes multiple fastening regions including a first fastening region in which an array of fastener elements including stems are arranged, the stems integrally molded with and extending from a resin base, and a second fastening region of fibrous loops releasably engageable with the fastener elements.

In some embodiments, the inflatable pad has a fitment allowing communication with the inflatable pad. The fitment can be formed to mate with a flexible hose for hydraulic communication with the pad.

In certain cases, the second fastening region overlaps the compartment.

The fibrous loops of the second fastening region can be formed of a dimensionally stable loop material that inhibits an increase in bounded area of the compartment during inflation.

According to another aspect, a method of forming an inflatable product includes overlapping two areas of flexible sheet material. The overlapped areas are bonded in selected regions to form a pouch defined between overlapped areas and bounded by the bonded regions, the pouch defining a limited opening for inflating the pouch. Both an array of fastener elements comprising stems integrally molded with and extending from a common resin substrate, and a region of fibrous loops releaseably engageable with the fastener elements are included on the sheet material.

In some embodiments, a valve is attached to the pouch at the limited opening to inhibit egress of fluid from the inflatable pouch through the limited opening, once inflated. The valve can include a passage that is collapsible in response to internal pouch pressure. Where the valve includes a collapsible passage, the valve can be formed of two sheets of flexible material sealed together in face-to-face relation along either side of the passage. In some cases, the valve includes a removable plug that can seal the opening.

In certain embodiments, the array of fastener elements are formed by introducing a molten resin to a gap defined adjacent a mold roll surface, the mold roll including an array of cavities for molding fastener element stems, the resin introduced such that resin enters the cavities to form fastener element stems while excess resin forms the substrate. In some of these embodiments, the sheet material is formed of the molten resin. In some cases, a preformed sheet material is introduced to the gap under conditions to bond the substrate to a surface of the sheet material. In certain cases, distal ends of the stems are post-formed to form engageable heads extending outwardly from the stems. In other cases, the cavities are shaped to form loop-engageable fastener elements. In some embodiments, a preformed loop material is introduced to the gap under conditions to permanently bond the loop material to the sheet material.

In certain embodiments, the method includes overlapping two different areas of the flexible material and bonding the different overlapped areas in selected regions to form a second pouch defined between overlapped areas and bounded by the bonded regions.

According to another aspect of the invention, a method of measuring blood pressure includes providing an unused blood pressure cuff formed by overlapped areas of flexible material forming therebetween an inflatable pouch, the pouch bounded by bonded regions of the flexible material and defining a limited opening for inflating the pouch. The flexible material includes multiple fastening regions including a first fastening region in which an array of fastener elements with stems are arranged, the stems integrally molded with and extending from a common resin substrate and a second fastening region of fibrous loops releasably engageable with the fastener elements. The blood pressure cuff is wrapped around a portion of a patient's body and the pouch is inflated to a pressure associated with blood pressure measurement. The blood pressure cuff is removed from around the portion of the patient's body and the blood pressure cuff is discarded.

In certain respects, the invention features inflatable products that can be made so lightweight and inexpensive as to be practically disposable, yet, in some cases, useful for performing critical medical functions. In this sense, their disposability can help to reduce the need for in-field sterilization of such components, and the risk of contamination. By configuring the fasteners to have particularly low peel resistance, the wraps can be removed without significant trauma to underlying tissue, making them particularly useful for wound and bum care. Many practical configurations of the wraps are readily manufacturable in continuous processes on standard bag-making equipment, and integrating the fastener components into the substrate of the wraps can significantly reduce the complexity and assembly costs of the overall products. Moreover, wraps can be produced with particularly low overall weights and material costs.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1A illustrates a pair of sheet material strips.

FIG. 1B is an embodiment of an inflatable product formed with the strip of FIG. 1A.

FIG. 1C is a side view of the inflatable product of FIG. 1B.

FIG. 1D is a side view of the inflatable product of FIG. 1B with an inflated pouch.

FIGS. 2A and 2B illustrate an embodiment of a self-sealing valve.

FIGS. 3 and 4 illustrate various methods of forming flexible sheet material suitable for forming inflatable products.

FIG. 4A illustrates an embodiment of an array of fastener elements extending from a common substrate and FIG. 4B is a detail view of a fastener element of the array of FIG. 4A.

FIGS. 5-5B illustrate a method suitable for forming an inflatable product.

FIGS. 6 and 6A illustrate another suitable method for forming an inflatable fastener product.

FIGS. 7 and 7A illustrate an embodiment of an inflatable product formed by the method of FIGS. 6 and 6A. FIGS. 8A and 8B show the inflatable product of FIG. 7 wrapped about an object.

FIG. 9 is another embodiment of an inflatable product.

FIG. 10 illustrates another embodiment of an inflatable product.

FIGS. 11 and 11A are front and side views, respectively, of another embodiment of an inflatable product.

FIGS. 12 and 12A are front and side views, respectively, of an embodiment of an inflatable product.

FIG. 13 is another embodiment of an inflatable product.

FIG. 14 illustrates the inflatable product of FIG. 12 wrapped about a patient's arm.

FIGS. 15 and 15A illustrate embodiments of an inflatable product.

FIGS. 16A-16C illustrate section views of various embodiments of inflatable products that include a compartment for holding an object.

FIGS. 17A and 17B illustrate an embodiment of an inflatable product wrapped about an ankle.

FIGS. 18-18C illustrate another embodiment of an inflatable product.

FIGS. 19-19C illustrate another embodiment of an inflatable product in the form of an inflatable bag.

FIGS. 20A and 20B illustrate a web of an embodiment of an inflatable product and FIG. 20C illustrates an inflatable product of FIG. 20 in use.

FIGS. 21 and 22 show inflatable product embodiments used as dunnage bags.

Like reference symbols in the various drawings indicate like elements.

### DETAILED DESCRIPTION

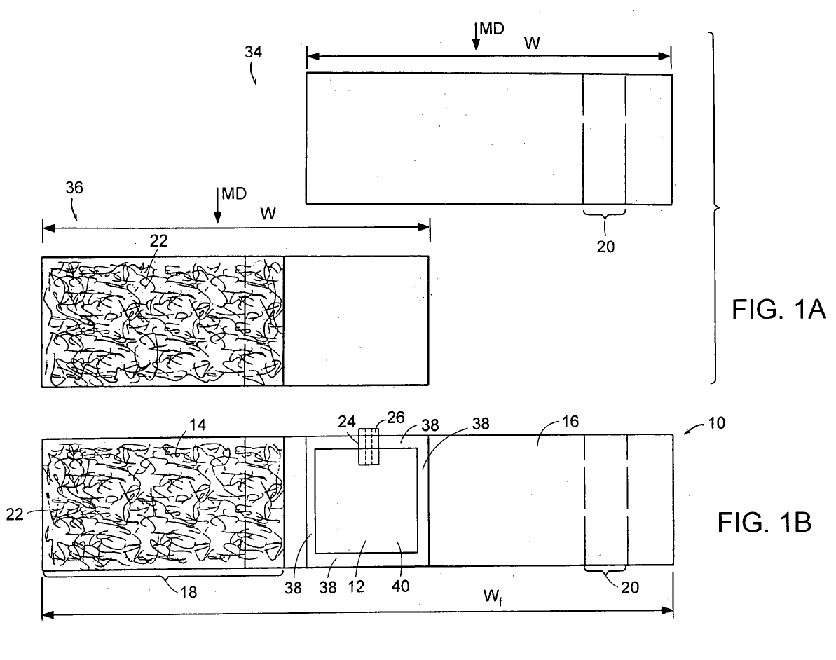

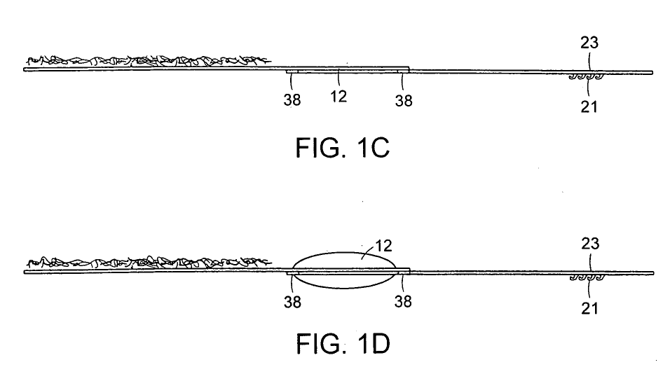

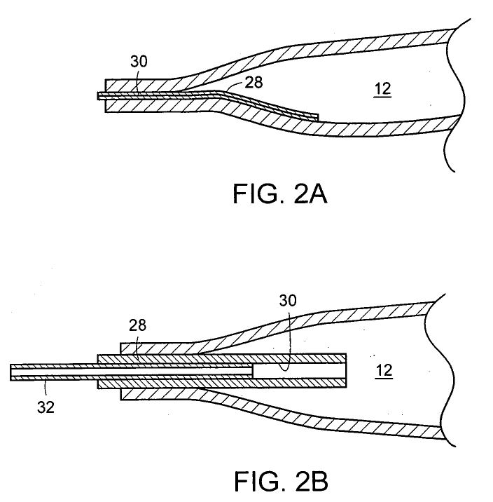

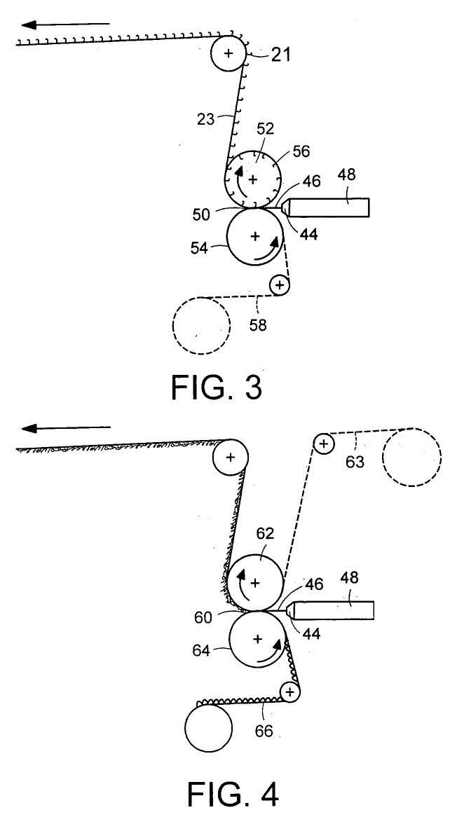

Referring to FIG. 1B, an inflatable product 10, such as for medical use, includes an inflatable pouch 12 (see FIG. 1D) and a pair of straps 14 and 16 extending outwardly from opposite sides of the inflatable pouch. Each of the two straps 14 and 16 include a fastening region 18, 20 with fastening region 18 containing engageable, fibrous loops formed of a non-woven material 22 and with fastening region 20 containing an array of loop-engageable fastener elements 21 (e.g., J-shaped hooks, L-shaped hooks, mushroom-shaped hooks, flat topped hooks) having stems extending from a common substrate 23 (see FIGS. 1C and 1D). The straps 14 and 16 are of such a length to wrap about an object (e.g., a portion of a patient's body) and to releasably engage the fastening regions 18 and 20 together (e.g., to hold pouch 12 snuggly against the object).

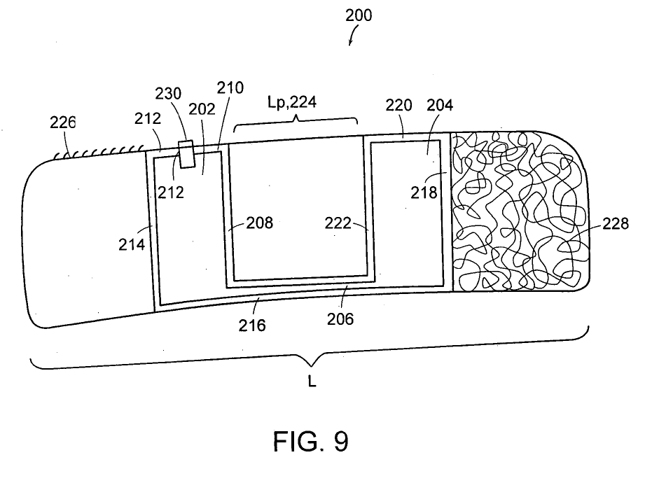

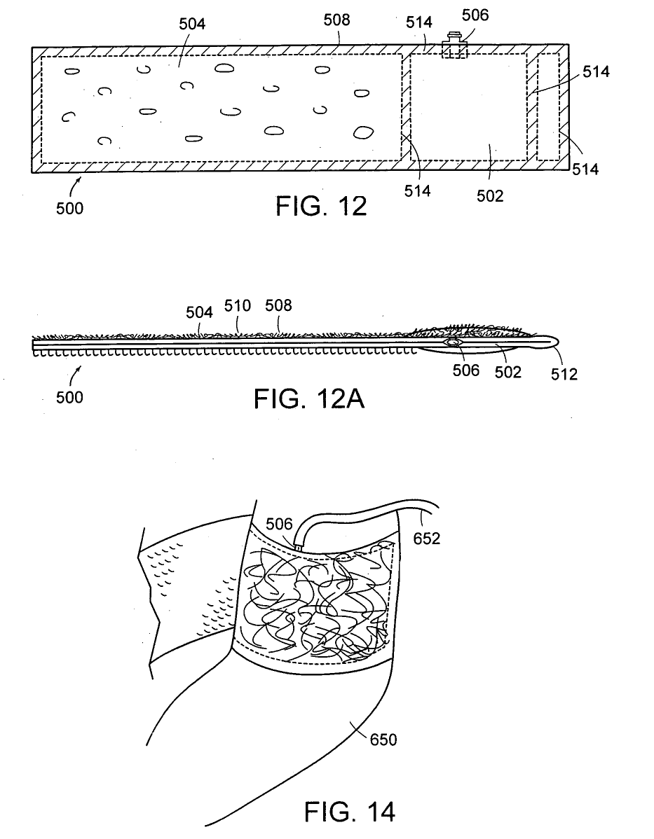

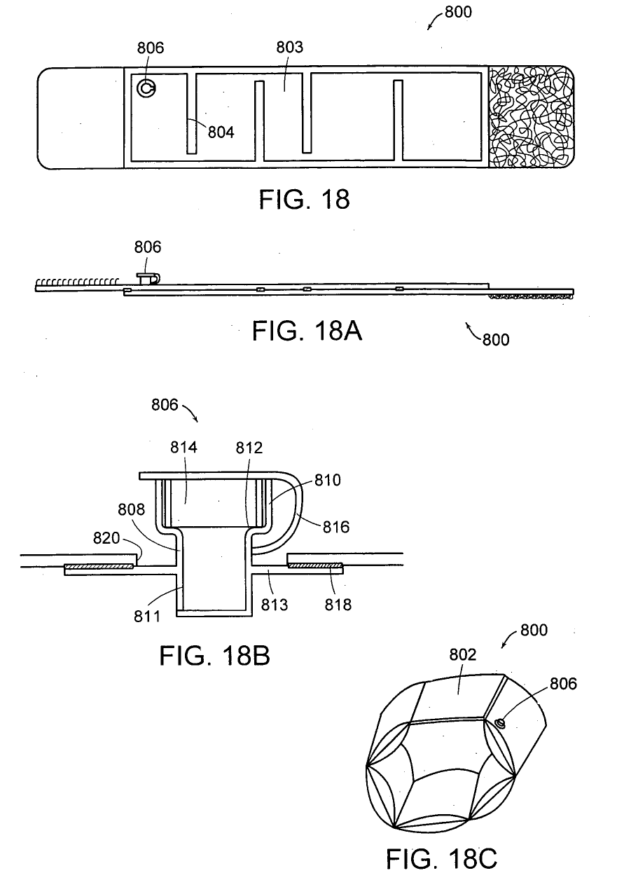

As shown, both fastening regions 18, 20 extend over only a portion of an associated strap 14, 16, positioned on either side of the pouch. Generally, however, the fastening regions can be sized and located as desired. In some embodiments, a fastening region extends over the entirety of a strap (see, for example, FIG. 11A). In some cases, a fastening region extends over the entire length of the inflatable product, overlapping the pouch (see, for example, FIG. 7). In other cases, a fastening region only overlaps the pouch (see, for example, FIG. 16A). In some embodiments, a fastening region extends from an edge of a strap and/or pouch.

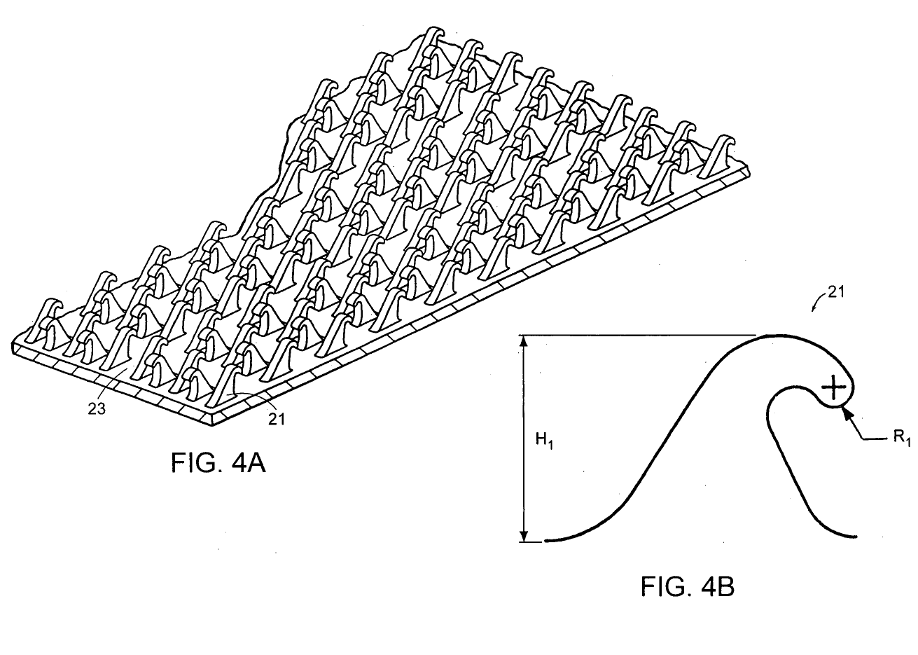

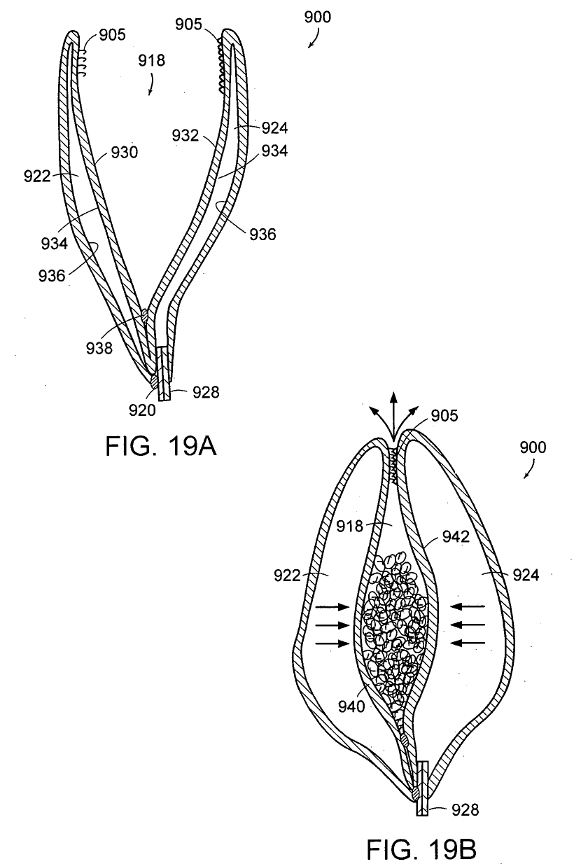

To inflate pouch 12 (e.g., prior and/or subsequent to positioning the inflatable product about an object), the pouch has a limited opening 24 through which a self-sealing valve 26 is inserted and securely bonded therein (e.g., by heat welding, ultrasonic welding, adhesives). Referring to FIGS. 2A and 2B, an example of a self sealing valve includes a female coupling member 28 (e.g., a collapsible tube) having a collapsible inner diameter 30 that can receive a male coupling member 32 (e.g., a relatively rigid tube). Referring to FIG. 2B, the pouch 12 is inflated by inserting the male coupling member 32 into the female coupling member 28 and forcing a fluid (e.g., air) through the male coupling and into the pouch until the pouch inflates to a desired level, such as a desired pressure or size. Once the desired level of inflation is reached, the male coupling member 32 is removed and the female coupling member 28 collapses to close the inner diameter. As shown by FIG. 2A, with pouch 12 inflated, pressure within the pouch forces the female coupling member 28 closed and against an inner surface 31 of the pouch to inhibit the flow of air out of the pouch. In some embodiments, the female coupling member has a “normally closed” configuration (e.g., by forming the female coupling member of an extensible material and/or closing down the inner diameter with an extensible fastening). In some embodiments, the female coupling member is formed of a pair of strips of flexible film (e.g., polyethylene) bonded together at edges to form a tube. By employing a self-sealing valve, the pouch can be relatively simply “freshened up” with additional fluid when desired. Other types of valves can also be used (e.g., manually sealing valves, such as a Boston valve; see, e.g., FIG. 18B).

Referring now to FIGS. 1A and 1B, pouch 12 can be formed by overlapping two strips 34, 36 of width W formed of a relatively thin (e.g., from about 0.0005 to about 0.008 inch thick, from about 0.001 to about 0.005 inch thick, such as about 0.004 inch thick), flexible material to form overlapped areas of the flexible material with the valve 26 positioned therebetween. To form the inflatable product 10 of width W_{f}, flexible material is bonded together (e.g., by welding, such as heat welding, ultrasonic welding, adhesives) in selected bonding regions 38 (see also FIG. 1C) forming a desired pouch design with the pouch 12 formed between the overlapped areas of flexible material and bounded by the bonded regions. In some cases, a lamination barrier, such as a varnish and/or overprint may be used to prevent bonding in selected regions. The flexible material is also bonded to the valve (e.g., about the valve's periphery), as noted above, to secure the valve within the limited opening 24 and also to form a fluid-tight seal to inhibit unwanted fluid leak. To facilitate bonding of the valve within the limited opening, the valve can be formed of a compatible plastic material that can be welded to the flexible material. Other techniques may also be such as an intermediate bonding layer formed of a material weld-compatible with both the valve material and flexible material.

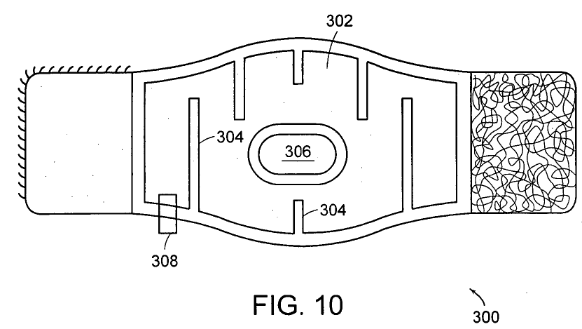

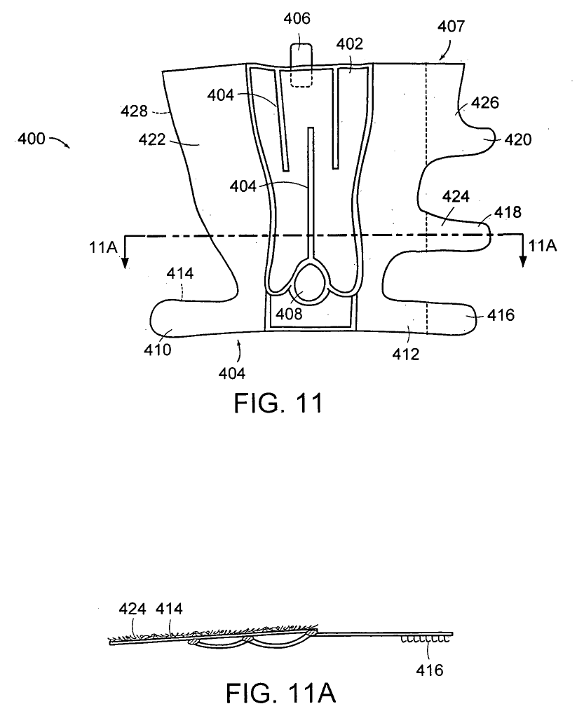

As shown, the pouch has only a single compartment 40. The pouch can include dividers (see, for example, FIG. 10), multiple compartments and, in some cases, the compartments are hydraulically interconnected (see, for example, FIG. 9), which can facilitate use of only one inlet for inflating all compartments of the pouch. As will be described in greater detail below, the pouch can also be formed using a single piece of folded flexible material and bonding the folded flexible material selectively within overlapped areas.

As can be seen by FIGS. 1C and 1D, the common substrate 23 from which stems of fastener elements 21 extend is formed by the flexible sheet material. Such a configuration can be advantageously formed using methods based on those described by Fischer in U.S. Pat. No. 4,794,028, the disclosure of which is incorporated herein by reference.

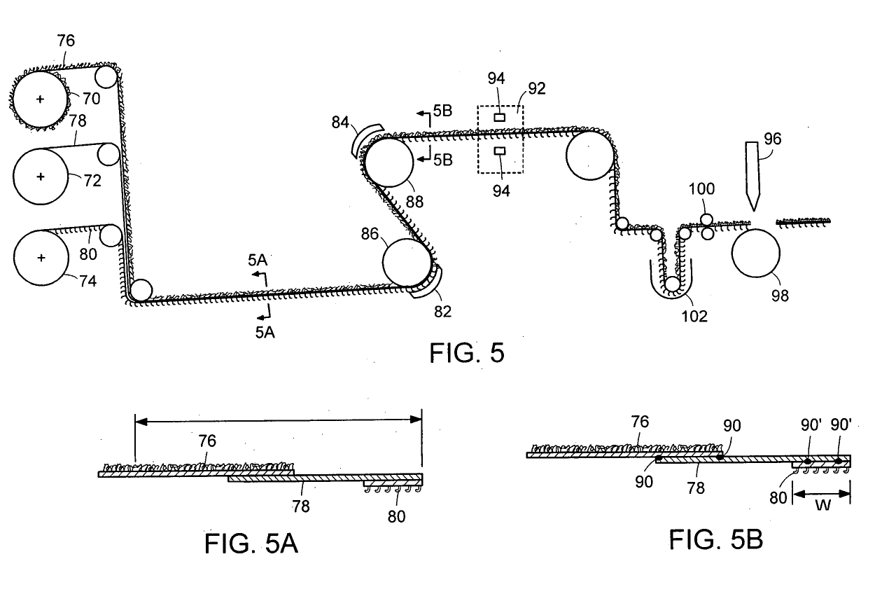

Referring to FIG. 3, a method for producing a continuous sheet of flexible material, e.g., useful for forming strip 34 of FIG. 1A, includes molding, by calender action, moldable resin 46 extruded through flat die 44 and provided by extruder 48. Extruder 48 supplies a continuous sheet of the molten resin 46 (e.g., polyester copolymer, nylon, polypropylene, polyethylene) to a gap 50 defined by a mold roll 52 and a pressure roll 54. Mold roll 52 contains an array of miniature mold cavities 56 extending inwardly from a peripheral surface of the mold roll 52 for forming loop-engageable fastener elements 21. As shown, the cavities 56 are shaped to form loop engageable fastener elements 21 (see FIG. 1C), but, in other embodiments, can be shaped to form stems or other hook preforms, self-engaging formations, or other fastener features. Methods of forming post-formed hooks from molded stems is described in further detail by Parellada et al. in U.S. Pat. No. 6,248,276, the contents of which is incorporated by reference as if fully set forth. As the resin 46 enters the gap 50, some of the resin is forced into cavities 56, forming fastener elements 21 due to the pressure in the gap and at the same time excess resin 46 forms substrate 23 from which the fastener elements extend.

As a variation, the common substrate 23 is bonded to a preformed sheet 58 (shown by dotted lines) of flexible material. In this variation, the preformed sheet 58 of flexible material is introduced to the gap 50 along with the molten resin 46 under conditions to permanently bond resin 46 to the preformed sheet. The resin 46 can be introduced as a relatively narrow band or intermittently as longitudinally discontinuous islands of resin. Multiple bands of resin can also be introduced. Introducing a preformed material while molding fastener elements to bond the preformed material to a resin base of the fastener elements is described in further detail by Kennedy et al. in U.S. Pat. No. 5,260,015, and in situ lamination of strips of molded hooks is disclosed in U.S. Pat. No. 6,205,623 by Shepard et al., the contents of which are incorporated by reference as if fully set forth.

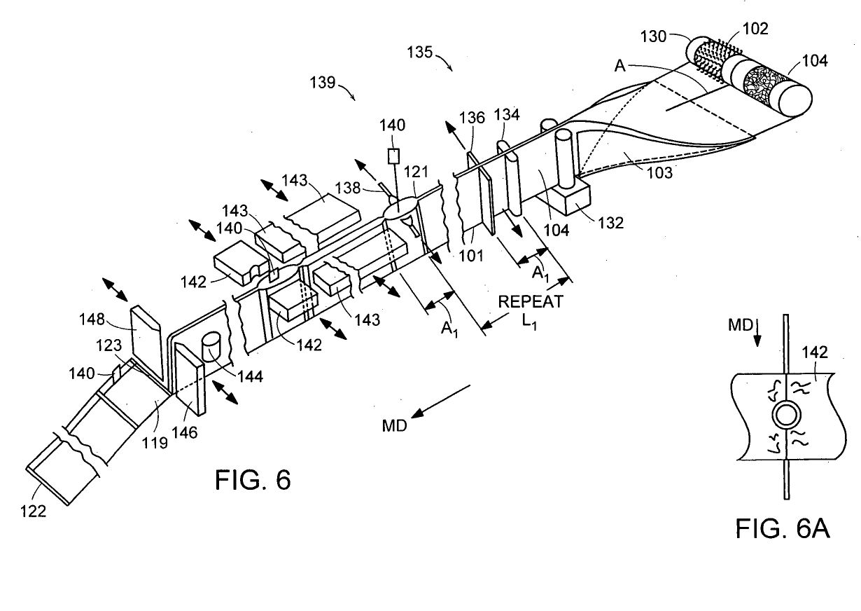

Referring now to FIG. 4, a method for producing a continuous sheet of flexible material, e.g., useful for forming strip 36 of FIG. 1A, includes joining a preformed loop material 66 (e.g., a non-woven material, a woven material, a knit material) to an appropriately selected region of an extruded sheet of molten resin 46 by introducing the loop material to a gap 60 formed between calender rolls 62 and 64. Also, or in the alternative, a preformed material 63 (shown by dotted lines), such as a loop material, may be introduced at the opposite side of the incoming resin 46 between the resin and roll 62. Bonding a preformed material to a resin sheet is described in further detail by Kennedy et al. in U.S. Pat. No. 5,260,015 (already incorporated by reference) and by Clune et al. in U.S. Pat. No. 6,656,403, the disclosure of which is hereby incorporated by reference as if fully set forth. Suitable loop materials include those disclosed in U.S. Pat. No. 6,342,285, and in the related PCT patent application PCT/US98/18401, filed as a continuation in part of the foregoing application, the entire disclosures of which are hereby incorporated by reference as if fully set forth.

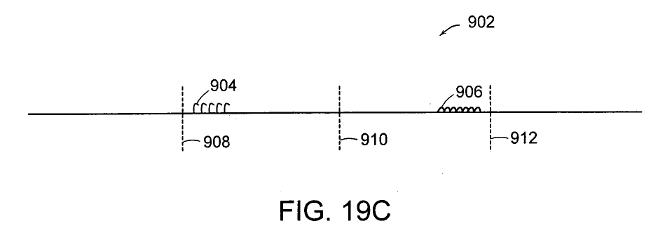

As an alternative to extruding molten resin, heat sealing, adhesive, or other joining processes may be employed to bond the preformed loop material 66 to a preformed sheet of flexible material, dependent upon the material and construction of the loop material and the required quality of the joint. Additionally, calender roll 62 may include molding cavities for molding projections that extend from a common substrate, as in FIG. 3, above. This can provide a flexible sheet material having both, e.g., loop-engageable and hook engageable fastener elements bonded to a common sheet of flexible material (see, for example, FIG. 19C).

As a specific example, shown by FIGS. 4A and 4B, the fastener elements 21 may be of molded form available from Velcro, USA under designation CFM29, shown magnified in FIG. 4B, having dimensions H₁=0.0149 inch and R₁=0.0015 inch. The loop material may, for instance, be non-woven hook-engageable material, available from Velcro, USA as loop L3310, formed according to techniques shown in U.S. Pat. No. 6,342,285, already incorporated by reference. In other cases of hook and loop construction, other low-cost hook forms and loop materials may be employed, for instance hooks formed by post-forming molded stems and loops formed by light weight, inexpensive knit materials, for instance knitted loop material having a weight of less than 4 ounces per square yard, preferably less than 2 ounces per square yard.

In these and other roll-forming arrangements, provisions may be included to impart cross-machine strength to the formed composite web. In some cases, this is provided by a cross-machine-strong preformed fastener material or its carrier. In other cases, a reinforcing scrim may be introduced to the roll-forming station in a manner by which the scrim is embedded in the sheet being formed. Examples are introduction of an open reinforcing scrim on the molding roll side of resin entering the forming gap through which the resin passes in entering the mold cavities, and co-extruding two layers of resin while interposing a running length of the scrim between the layers before the layers enter the forming gap. The coextruded resin may be of the same or compatible materials. In one case a relatively stiff resin is employed to form the hooks and a thin upper part of the base layer, and a compatible resin, for instance a copolymer or blend, having elastomeric properties may form the predominate thickness of the base layer under the hooks and a calendered sheet extension as well. In this manner a wrapping material with elastic properties is formed. In some cases the reinforcing material may be omitted. In other cases the predominate thickness of the base layer may instead be selected for its toughness and the reinforcing layer may be omitted.

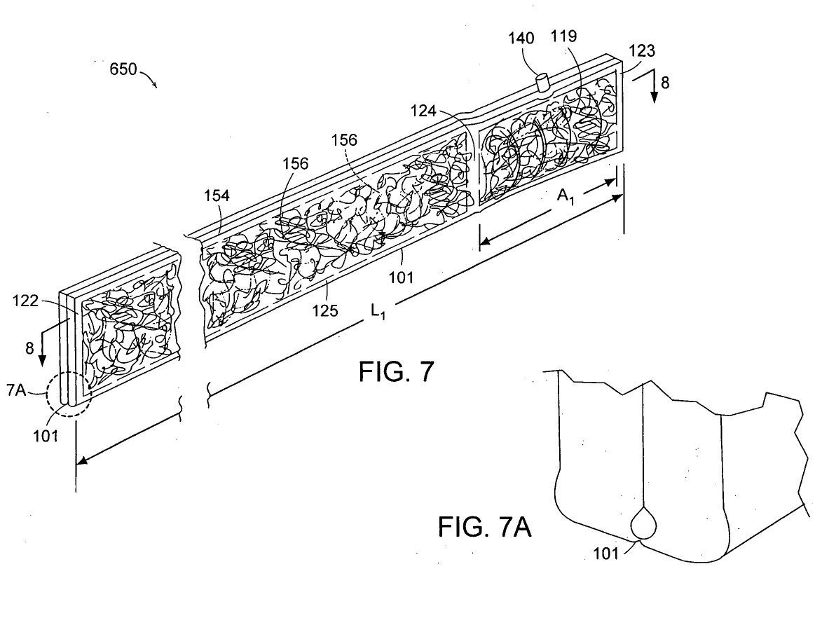

FIGS. 5, 5A and 5B illustrate an apparatus and process for joining multiple preformed sheets, such as those formed by methods described above, using common flat bag making machinery. At the beginning of the machine there are 3 supply rolls 70, 72, 74 of continuous weld-compatible materials. Roll 70 carries non-woven loop material 76 with thermo-plastic backing of flexible material (or there can be a fourth roll that carries a sheet of flexible material that is to be bonded to the loop material), roll 72 carries back sheet 78 of flexible material, and roll 74 carries hook material 80. The webs from supply rolls 70, 72 and 74 are overlapped and combined into a tri-component continuous web as shown in FIG. 5A, employing thermal sealers 82 and 84 (e.g., a drag sealer). The sealers include a nip formed by a round drum 86 or 88 and a heated drag profile, e.g., a member which slidingly engages the web with heat and pressure along a desired weld line. The heat, time, and pressure of the drag through the nip are requested to join two sheets through thermal welding.

Loop drag sealer 82 seals through the back sheet 78 into the backing of loop material 76 creating a pair of thermal seals 90 that joins webs 76 and 78 in selected bonding regions.

Hook thermal drag sealer 84 seals the continuous hook material 80 to combined webs 76 and 78. Two seal welds 90′ are provided (see FIG. 5B), one on each edge of the hook material 80.

The combined web of material is directed to station 92 where a valve (e.g., a self-sealing valve) formed of a weld-compatible material is inserted between the loop material 76 backing and the back sheet 78. To form a pouch, a set of weld bars 94 weld the loop material and back sheet forming bonding regions that extend between seal welds 90. Weld bars also weld the valve to the loop material and backing. Due to the position of the valve and shape of the bars, a limited opening is formed that can be used to inflate the pouch.

A heated cut-off blade 96 severs the sheet against round anvil 98 transversely to machine direction at running length intervals. A servo drive mechanism 100 moves the sheets along until cutoff blade 96. The take-up dancer assembly 102 provides web inventory or film inventory so that the materials can progress with continuous motion past the drag sealers and with rapid intermittent motion the high temperature, radiant heat cut-off cycle.

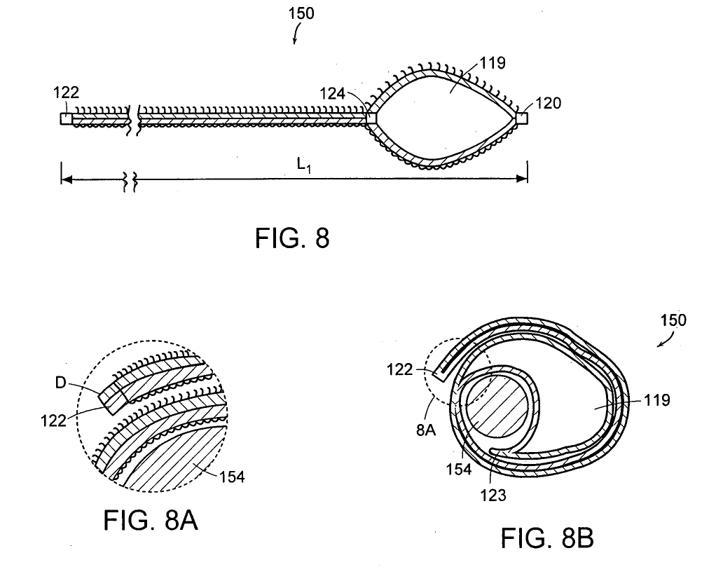

As an alternative to bonding separate sheets, suitable methods can include overlapping (e.g., by folding) and bonding a single sheet of flexible material that carries regions of engageable fastener elements in selected bonding regions. Referring now to FIG. 6, the operative components of a horizontal poucher machine 110 are shown that have been specially adapted to form extended, self-engaging inflatable products (see FIG. 7). The poucher extends from a supply roll 130 of the preformed material to a cutoff blade 146. In this example, a specially prepared, continuous composite component, e.g., produced according to FIG. 4 that includes a mold roll with fastener element cavities, is led from the supply roll 130 into a former 105 where the composite is shaped to be folded to form overlapped areas of the composite material. To facilitate folding, the composite sheet can include a folding formation (FIG. 7A), such as a narrow region of reduced thickness located between the regions of fastener elements. Once centered on former 105, the composite passes through drive stations 132 and 144 located near the entry and exit of the machine, respectively, and which are coupled to act in unison. Drive station 132 include rolls extending the full width of the composite material to pull the sheet-form composite through the former 105. In coordination with the downstream drive station 144, drive station 132 also tensions the material.

By indexing action, the sheet material passes from former 105 to a pair of sealing stations 134 and 136 where sealing bars 137 bond regions 121 and 124 of the folded sheet material together. In some embodiments, the sheet material includes weld flanges free of fastener elements to facilitate bonding. After bonding, the sheet material passes from the weld stations 134 and 136 to an insertion station 139. Here, the bonded regions 121 and 124 are positioned on opposite sides of a pair of oppositely acting separators, e.g. suction cups 138. Suction cups 138 engage respective sides of the web at between the bonded regions 121 and 124 and by moving in opposite directions away from each other, the top of the pouch between bonded regions 121 and 124 is opened to enable placement of a valve 140 between the overlapped areas at an opening between the bond regions 121 and 124.

One alternative method for opening the sides of the pouch is to use hook and loop fasteners to engage and pull back the loop and hook sides of the material, respectively. This can be used to open the sides slightly, at which time spreader blades can be inserted and spread apart to complete the action. Another method for opening is to place the fold axis A slightly off center of the overall web width. Thus, when folded about axis A, one edge of the folded material will extend higher than the other. Given height difference between opposed edges, high-pressure air blown into the pouch area or mechanical means such as pinchers can open the sides.

With the valve 140 held in place at the opening, downstream drive station 144, in conjunction with drive station 132, indexes the folded and welded material from the insertion station 139 to top seal station 143. Sealing jaws of station 143 with motion similar to that of the heat seal bars at stations 134 and 136, move in to seal and out to release. While the machine is forming the bonded regions at station 135, and inserting valve 140 at station 139, the sealing jaws at station 143 engage to seal shut the top of the inflatable product and form weld 115. Simultaneously, the sealing jaws at station 142 engage to form weld 113 and to secure the valve 140. As above, the overlapped areas are bonded such that a limited opening is formed to allow for inflation of the pouch (see FIG. 6A). Meanwhile, the downstream cutting jaws 146, 148 sever the bonded, overlapped composite at bonded region 121 to form trailing weld portion 123 of the leading unit and leading weld portion 122 of the next unit. This severs the leading wrapping from the continuous assembly.

The repeat length L₁ for the system illustrated is established by the desired length of the conformable self-securing inflatable product and extends from sealing bar 134 to previous weld formed by that bar at the upstream end of filling station 139. This repeat length is adjustable between production runs by adjusting machine index, to determine the length of the self-securing wrapping being produced. The distance between seal jaws 134 and 136 is also adjustable along the length of the machine, the spacing depending on whether larger or smaller pouch space is desired, based upon volume or size of the contents to be inserted. Additional heat seal jaws of any suitable shape and/or size may be provided to form multiple pouches or compartments within a single pouch, or to locate the pouch at the center (see, for example, FIG. 1B) or other desired location of the inflatable product, or to provide optional cut lines at which users may shorten the product as with a scissors or portable knife blade.

At the end of the pouch forming line, knife edge 146 cuts the product against anvil 148, to sever the leading wrapping unit. The finished unit 119 shown falling off after cutting, may instead pass to a shingled inching conveyor (not shown) from which it is removed and packed in cases. Standard production throughput may be of the order of 100 to 200 units per minute, in many cases the limiting factor is how fast the contents may be placed in the pouch.

Since the machine has a start/stop operation, with dwell time required, a conventional provision such as an accumulator may be provided to provide a constant turning of the supply roll.

During the fold, seal, fill, and cut sequences the continuous composite sheet advances in-line in continuous sheet form from roll 130 with hook and loop bands of the preformed composite material facing downward. As the composite material moves from supply roll 130 to drive station 132, it folds along the machine direction about the fold axis A so that the plastic backings of the sides are brought face-to-face, positioned to be heat sealed together, while the hook and loop panes are caused to lie on opposite outside faces of the unit. The sides are heat sealed transversely to machine direction at sealing stations 134, 136 by heated heat seal bars that move in, seal and return in a forming cycle, the composite stops. The seal 121 formed by heat seal bar 134, being twice as wide as the seal 124 formed by bar 136, enables the final cut at blade 146 to bisect that seal to form trailing weld 123 of the leading wrapping unit and leading weld 122 of the next unit. The pouch 119 for each unit is, formed by the two transverse heat seals 123 and 124, top machine-direction seal 113, if employed, and the bottom fold 101 of the material, (and additional bottom seal 125 when employed). Each time the web stops at the insertion station, an appropriate separator separates the sides of the pouch sufficiently to enable placement of the valve 140. The final drive station 144 has specially shaped drive elements to engage and drive the composite while accommodating passage of the valve. In one case, as shown, a short drive pair engages a mid region only of the formed composite to pull the material through the machine and advance it to the cut-off station.

Referring to FIGS. 7 and 7A, an inflatable product 150 formed by the above-described process includes an inflatable pouch 119, valve 140 and a flexible, elongated strap 154 extending from the pouch. Inflatable product 150 is of selected length L₁ to match and exceed the girth of, e.g., the ankle, knee, torso or other part of the body or other object to be wrapped. Each face 156 of the inflatable product is entirely covered by an associated region of fastener elements, except along its weld lines. The plastic backings of both faces enable welding together along selected weld lines (forming welds 123 and 124). In some instances, a bottom weld 125 parallel to top welds 113, 115 is formed immediately above the fold 101.

Referring to FIGS. 8A and 8B, the inflatable product 150 securely holds the inflated pouch 119 close to an object 154, for instance an ankle or arm. The inflatable product 150 fastens to itself by engaging the loop fastening region 156 with the hook fastening region 158 (see FIG. 8B). Because of the weld 122 provided at the free end of the wrapping shown in FIG. 8A, a small dead region D of fastener material is provided that is incapable of fastening engagement. This provides an easy peel, free standing tip to grasp to initiate unwrapping. Thus, while the entire length of excess wrapping tightly engages itself without an undesirable loose tail, the extreme tip D remains free to be grasped.

Generally, the bonding regions, fastening regions and overlap areas of flexible material can be selected, sized and/or shaped as desired depending on the end use of the product. Referring now to FIG. 9, as an example, an inflatable product 200, such as for use as a splint (e.g., for an injured hand), includes a multi-compartment configuration where a first compartment 202 is hydraulically connected to a second compartment 204 by a passageway 206 spanning a distance Lₚ between the two compartments. The passageway 206 and compartments 202, 204 are bounded by bonded regions 208, 210, 212, 214, 216, 218, 220 and 222 of flexible material. Passageway 206 is sized to allow a user (e.g., a patient, nurse, doctor) to inflate (e.g., by mouth) each of the first and second compartments 202, 204 through a valve 230 positioned within a limited opening 212 formed by the first compartment 202.

By spacing the compartments 202 and 204 from one another, a somewhat flexible, non-inflatable region 224 is formed that allows the inflatable product 200 to be folded within the region 224, between the relatively rigid, inflated compartments to be wrapped about an object, such as a hand, preferably sandwiching the object between the two inflated compartments (e.g., for protection, immobilization). The length L of the product is sized to wrap about the desired object and releasably fasten by bringing fastening regions 226 and 228 together.

FIG. 10 shows another example of an inflatable product 300 in the form of a splint for a hand. The inflatable product includes an inflatable pouch 302 having multiple dividers 304 that interrupt space within the pouch. The dividers 304 are formed by bonding selected regions of overlapped flexible material such that the rigidity of the inflated pouch is affected to allow the inflatable product to extend about the hand and releasably fasten. As can be seen, the dividers 304 are positioned to form a tortuous path for fluid to travel as the pouch is inflated using valve 308. The inflatable product 300 includes a hole 306, through which the thumb can be inserted.

Referring now to FIGS. 11 and 11A, an inflatable product 400 in the form of a leg and/or ankle splint includes an inflatable pouch 402 having multiple dividers 404 that interrupt space within the pouch to adjust rigidity of the inflated pouch, as described above. Referring to FIG. 11, a valve 406, located at a top 407 of the splint, provides access to the interior of the pouch for inflation. Located near a base 409 of the splint is a hole 408 into which a heel of a foot can be placed.

To fasten the inflatable product 400 about the leg and foot, a wearer's ankle is first placed within hole 408 with the wearer's toes pointing directly away from valve 406. Straps 410 and 412 can then be brought together and fastening regions 414 and 416 releasably engaged about a top of the wearer's foot. The top 407 of the inflatable product can be raised such that pouch 402 abuts the wearer's calf. Straps 418, 420 and side 422 can be brought together and fastening regions 424 and 426 can be engaged with fastening region 428 about a front of a wearer's leg (e.g., at the shin).

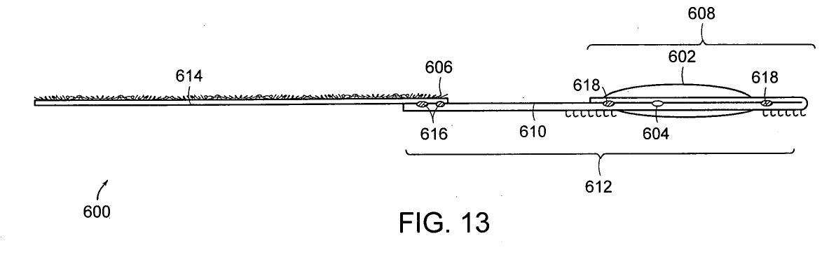

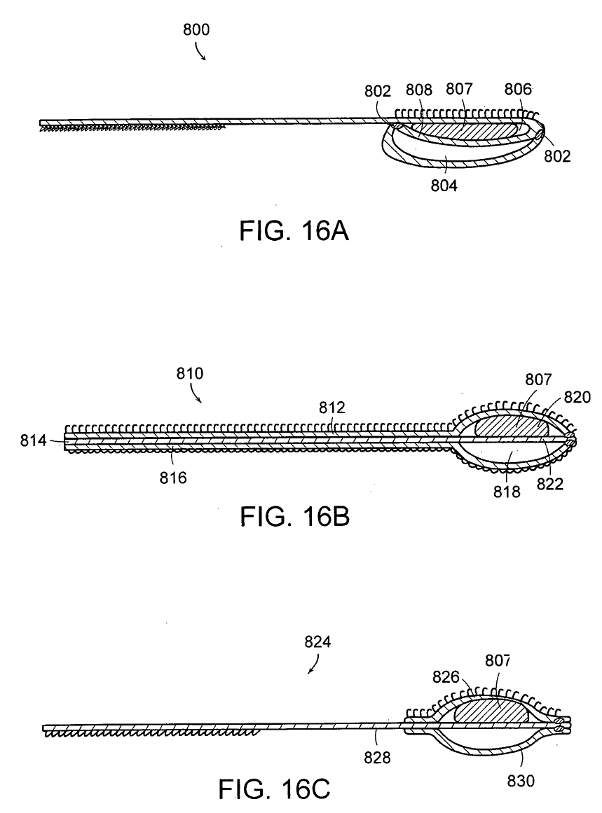

Referring now to FIGS. 12 and 12A, an inflatable product 500 in the form of a blood pressure cuff includes an inflatable pouch 502, a strap 504 extending from the pouch and a fitment 506 capable of coupling with a flexible tube (see FIG. 14) that is connected to a pump (not shown). In some embodiments, the inflatable product 500 includes more than one fitment, such as two fitments. As can be seen in FIG. 12A, a fastening region 508 of loops 510 extends over a large majority of the strap 504 and overlaps the inflatable pouch 502. In a preferred embodiment, the loops are formed of a dimensionally stable (e.g., non-extensible) loop material that inhibits or even prevents an increase in contact surface area between the pouch 502 and the patient's body as the pouch is inflated.

Referring still to FIG. 12A, the inflatable product 500 is formed by a single, folded strip 512 of flexible material that is bonded at select bonding regions 514 (FIG. 12) within overlapped areas of the flexible material. The inflatable product 500 can also be formed by overlapping separate strips of flexible material.

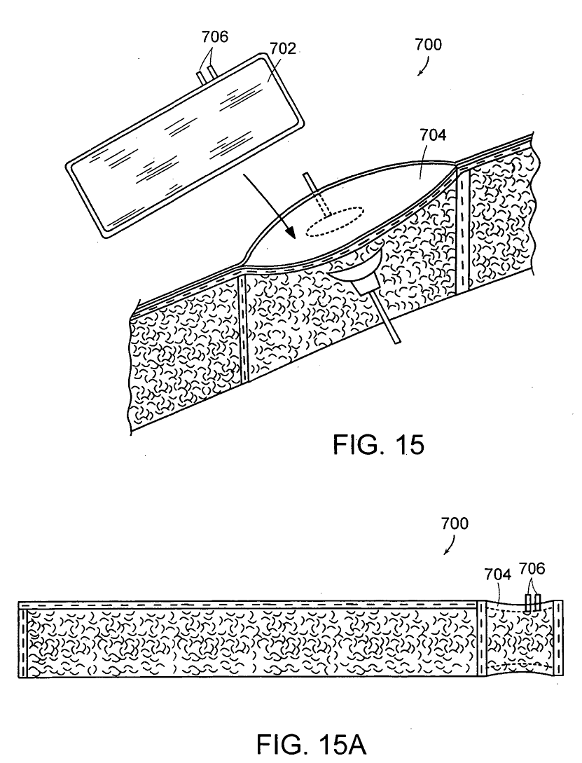

Referring now to FIG. 13, another embodiment of an inflatable product 600 suitable for use as a blood pressure cuff includes an inflatable pouch 602, a fitment 604 and a strap 606 extending from the inflatable pouch. The pouch 602 is formed by a fold 608 formed from a single strip 610 of flexible material, the fold only partially overlapping a remaining portion 612 of the strip Within the overlapped areas, the flexible material is bonded within select bonding regions 618, the pouch being bounded by the bonded regions. The remaining portion 612 is partially overlapped by a strip of loop material 614 and welded thereto by a pair of welds 616 to form strap 606.

To measure blood pressure, with reference to FIG. 14, a user wraps the wrapping around the upper portion of the arm 650 in the usual way of a blood pressure cuff. A flexible tube 652, mated with the fitment 506, is used as a conduit extending from a pump to the inflatable pouch 502 to pump the inflatable pouch with air. This tightens the wrapping until blood flow is constricted, and enables measurement of blood pressure during gradual release of air from the pouch 502 in the usual manner.

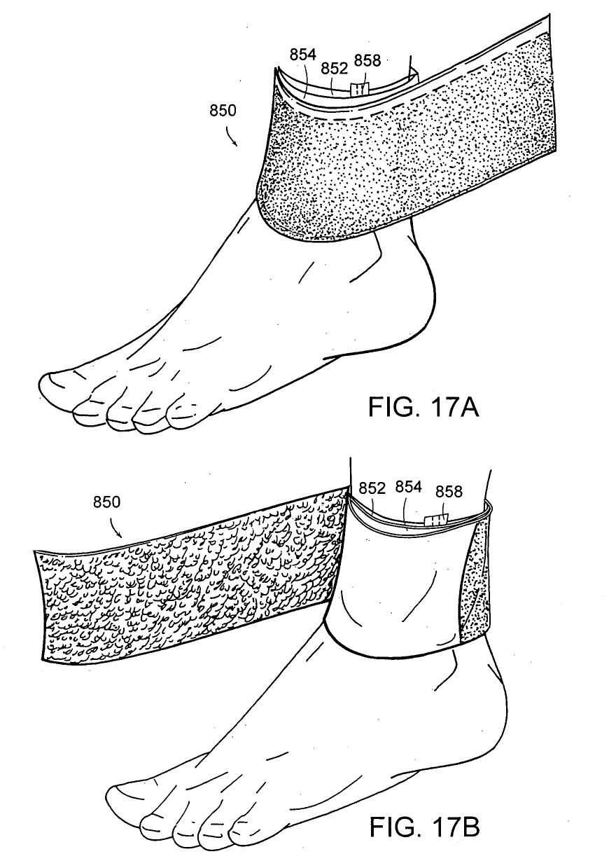

Referring to FIGS. 15 and 15A, a blood pressure cuff 700 includes an air pad 702 inside a compartment 704. During manufacture of the blood pressure cuff, e.g., as discussed in relation to FIG. 6 and partially shown by FIG. 15, a preformed air bladder is inserted into the compartment 704 with air tube endings 706 protruding -from an opening at the top, as illustrated by FIG. 15A. The top of the compartment 704 is not sealed after the air pad 702 is inserted. With appropriate selection of materials, the air pad 702 can be reused while the rest of the blood pressure cuff, intended for one time use, is disposable. This product is useful as a constricting or supporting device For instance, to measure blood pressure, the user wraps the blood pressure cuff 700 around the upper portion of the arm in the usual way of a blood pressure cuff. One of the tube endings 706 is used to pump the air pad with air. This tightens the blood pressure cuff, constricts blood flow and enables measurement of blood pressure during gradual release of air via one of the tube endings 706 in the usual manner.

FIGS. 16A-16C illustrate various inflatable product embodiments that also include a compartment (e.g., for carrying an object 807). Referring to FIG. 16A, a single strap of flexible material is folded along two fold lines (sometimes referred to as a “z-fold”) forming overlapped areas of flexible material and bonded at selected bonding regions 802 forming an inflatable product 800 having an inflatable pouch 804 and a compartment 806, each sharing a common wall 808.

Referring now to FIG. 16B an alternative design for an inflatable product 810 includes three strips of material 812, 814 and 816. Strips 812 and 816 overlap strip 814 forming areas of overlap that are bonded at selected bonding regions to form both an inflatable pouch 818 and a compartment 820, each sharing a common wall 822. FIG. 16C shows another design for an inflatable product 824, similar to the inflatable product 810 of FIG. 16B where strips 826 and 830 are shorter than strip 828.

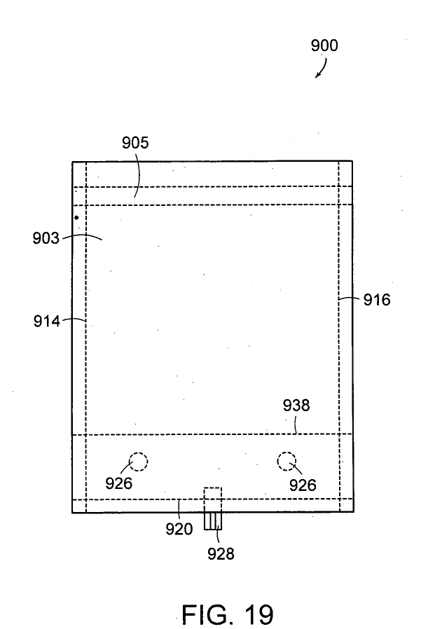

FIGS. 17A and 17B illustrate an inflatable product 850, similar to those described above, having both an inflatable pouch 852 and a compartment 854 positioned about the leg 856 of a patient. The wrap is positioned about the leg such that the inflatable pouch 852 with valve 858 positioned between the compartment 854 and the patient's skin. This can be particularly advantageous where a thermal pad is positioned within the compartment 854. By inflating or deflating the inflatable pouch, the heating/cooling effect of the thermal pad can be controlled. The compartment can also be placed directly against the skin.

For low cost inflatable products that include compartments for holding an object, such as those described above, it is advantageous that the compartment contents are inserted during manufacture of the inflatable product. This is done while top edges of the compartment are unsealed. The nature of the contents and their method of insertion depends upon the desired function. In cases that a preformed flexible package or device is to be inserted into the compartment, the compartment may thereafter remain open, or it may be sealed. If chemical reactants or drugs are introduced into the compartment, the edges of the compartment are sealed together with precision to provide a fluid-tight reaction chamber or protective compartment capable of safely holding the chemical reactants or drugs. By making the film of in situ laminate of resin capable of strong continuous heat seals, such as polyethylene, the flanges can be heat-sealed to each other to provide water tightness and strength along the edges as well as along the compartment walls. This ensures containment of the chemical reactants. An advantageous resin for such embodiments is commercially available linear, low-density polyethylene such as LL-6407 Exxon Mobile resin.

In instances in which the compartment is left permanently open, e.g. to enable the user to remove a cold pack, hot pack or other device and replace it with another pack of the same or different finction, closing flaps may be formed of the composite material and suitable strips or spot regions of surface fasteners are provided to close the compartment and secure its contents, but enable it to be opened and closed for reuse.

For many functions performable by the inflatable products conventional materials are useful. Typical reagents for a chemically-activated cold pack are ammonium nitrate and a separate, rupturable packet of water. To activate the cold pack, the water packet is broken by the user, initiating reaction of the water and ammonium nitrate as an endothermic reaction that creates a useful cold surface. In other cases a cooling fluid, gel or loose material is provided in a separate packet or in the compartment defined by the inflatable product. Such materials may be pre-cooled by the user by storage in a freezer, or a regulatable thermo-electric cooler may be inserted in the compartment (e.g., for moderate, continual cooling).

An insertable hot pack may contain material that can be heated by a microwave oven, e.g. a packet containing microwavable gel or heatable granules, or the compartment of the inflatable product may contain the particles, and the entire unit may be placed in the oven. An exothermically reactive mixture may be provided such as are employed with hand warmers, e.g. iron particles with carbon and salt; when exposed to air, the salt absorbs moisture, initiating oxidation of the iron particles and production of heat. A regulatable thermoelectric heater may be inserted in the compartment.

As previously noted, by selecting the location for the end cut of the inflatable product, a conformable, function-delivering inflatable product of any desired length may be manufactured. A inflatable product longer than that normally required to wrap around the part of the body, such as an ankle, is useful to cover a range of sizes or enable other uses. Since the surfaces of the inflatable product can be continuously covered by engageable fastening materials, the excess length may be wrapped upon and secured to itself, avoiding loose ends. A universal, long inflatable product can be usefully formed, to wrap around numerous components of the body, for instance the ankle, elbow and knee, even the head, of children, youth and adults. Likewise an extended veterinarian inflatable product may be provided for use with a range of sizes of animals. As previously suggested, the inflatable product may usefully be provided with a number of spaced apart transverse weld lines at which it may be selectively cut to shorten the inflatable product to the length required. In other cases, as in a hospital or emergency setting, a continuous roll of the conformable inflatable product with a sequence of repeating, spaced apart compartments and cut lines is provided from which a conformable, self-securing inflatable product of any desired length may be taken. Tear strips at the cut lines may be provided, that enable the inflatable product to withstand the desired tension during use, but which are readily severable as by provision of a special tear string, these are useful for shortening the inflatable product and for removing and disposing of the inflatable product in cases in which the fasteners are not readily releasable.

In the case of a hot pack, if the inflatable product is made with sufficient length, one can wrap it from the backside, around the waist of a person, to apply heat to the lower back. A typical dimension for L₁ to be useful about the ankle, knee and the head is in the range of 26-28 inches. About the waist, a length of 50 or 60 inches can be appropriate. For applications to the back by a user without assistance, the compartment is advantageously placed in the middle of the inflatable product. This enables attachment of the inflatable product while achieving proper placement of the treatment zone in the middle of the back.

In advantageous embodiments, fibrous loop material is so located in the inflatable product as to lie against the skin when the inflatable product is in place. This can provide soft, smooth comfortable contact. Such loop material can be of materials selected to act as a slight thermal barrier to prevent uncomfortable contact of cold or hot plastic directly against the skin.

The inflatable product may be constructed to be drawn tightly and secured, in a manner to produce compression against a desired region to be treated. Such snug cinching can ensure that the pack or instrument is intimately associated with the region to be treated for thermal therapy, drug delivery, etc. For a cold or hot pack applied e.g. to the ankle or knee of a person, for instance, the inflatable product is constructed to withstand a cinching force of between about 3 and 6 pounds.

Furthermore, a dressing, pack or instrument inserted in the compartment may be configured to provide a bulge in the inflatable product of desired form, shaped to produce a desired degree of localized pressure to enhance intimate contact with the region to be treated or to provide desired support.

In advantageous embodiments, fibrous loop material is so located in the inflatable product as to lie against the skin when the inflatable product is in place. This can provide soft, smooth comfortable contact. Such loop material can be of materials selected to act as a slight thermal barrier to prevent uncomfortable contact of cold or hot plastic directly against the skin.

In some embodiments, particularly those used for medical purposes, a transverse peel force required to release engagement between the fastening regions is relatively low (e.g., preferably less than about 0.2 pound per lineal inch and even more preferably less than about 0.1 pound per lineal inch). By providing a closure that requires a relatively low peel force for disengagement, potential for additional trauma due to removal of the inflatable product can be reduced.

Referring to FIGS. 18-18C, an inflatable product 800, such as for use as a floatation device (e.g., for children), includes an inflatable pouch 803 having multiple dividers 804 that interrupt space within the pouch. The dividers 804 are formed by bonding selected regions of overlapped flexible material such that the rigidity of the inflated pouch is affected to allow the inflatable product to extend about, e.g., an arm and releasably fasten (see FIG. 18C). As can be seen, the dividers 804 are positioned to form a tortuous path for fluid to travel as the pouch is inflated using valve 806.

Referring to FIG. 18B, the valve 806 includes a tube 808 with a first section 810 of a first diameter and a second section 811 of a second diameter that is less than the first diameter, forming a seating surface 812. Extending from the tube 808 is a weld flange 813 that is bonded by welds 818 to an inner surface of the flexible material at a limited opening 820. To close the valve 806, a plug 814 is inserted into an open end of the tube 808. The plug 814 is connected to the tube 808 by a connector 816. In some embodiments, tube 808 may have an inner and/or an outer threaded surface and plug 814 (or a cap) may have a matable threaded outer (or inner) surface. Valve 806 is sometimes referred to as a Boston valve. Other valves can also be used, such as self-sealing valves.

Referring now to FIGS. 19-19B, an inflatable product 900 in the form of an inflatable bag having a polyethylene body 903 welded to a closure strip 905 across its openable end. Closure strips for use with bags are described in greater detail by Clune et al, in pending U.S. patent application Ser. No. 10/357,608, the disclosure of which is hereby incorporated by reference as if fully set forth.

Referring also to FIG. 19C, the inflatable product 900 is formed from a single sheet 902 of flexible material (e.g., polyethylene) that carries a first fastening region of loop-engageable hooks 904 and a second region of hook-engageable loops 906. The sheet is folded along three fold lines 908, 910 and 912, creating inner overlapped areas 930, 932 and outer overlapped areas 934, 936 (FIG. 19A). Referring to FIG. 19A, the inner overlapped areas 930 and 932 are joined along bottom weld line 938. Both inner and outer overlapped areas 930, 932, 934 and 936 are joined along side edge lines 914, 916 and bottom edge line 920 forming an openable compartment 918 and two inflatable compartments 922 and 924 that are hydraulically connected by holes 926 already formed in the sheet 902. Extending from the inflatable product is a valve 928 (e.g., a self-sealing valve) for inflating the inflatable compartments 922 and 924. In some embodiments, the closure strip(s) can be applied subsequent to forming the compartments.

FIG. 19B shows the inflatable product having material placed within compartment 918. Compartments 922 and 924 have been inflated and expanded forming a protective casing extending about inner compartment 918. Air pressure applied against inner compartment 918 walls 940 and 942 forces air from the inner compartment 918.

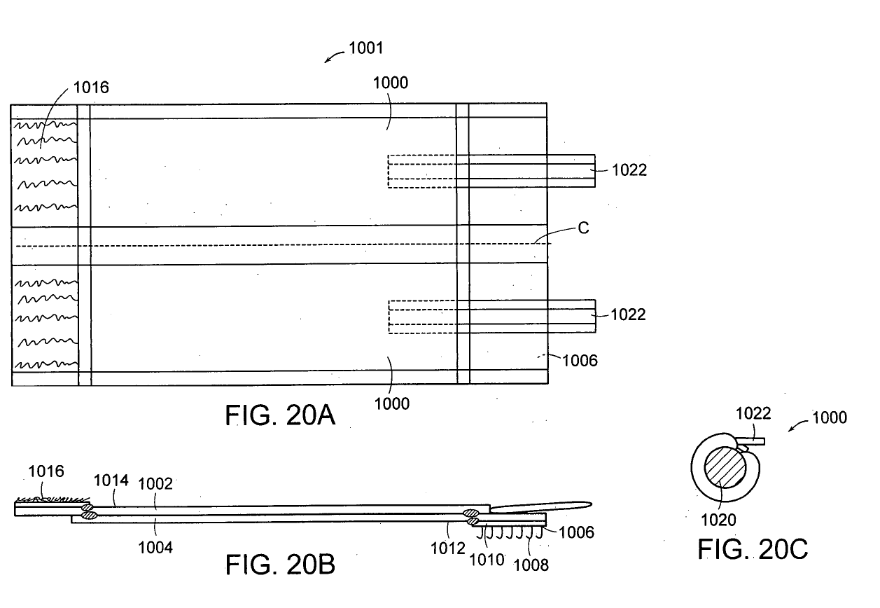

Referring to FIGS. 20A-20C, an example of a preform web 1001 of inflatable products 1000 is shown. Referring to FIG. 20B, the web is formed of two overlapping sheets 1002, 1004 of flexible film (e.g.; polyethylene). A band of hook closure strip 1006 having an array of hooks 1008 extending from a common substrate 1010 is bonded to a surface 1012 of sheet 1004 and a band of loop material 1016 is bonded to a surface 1014 of sheet 1002 such that the fastener elements of the two bands 1006 and 1016 extend from opposite sides of the web. The sheets are bonded within the overlapped areas at selected bonding regions to form inflatable pouches. A self-sealing valve 1022 provides access to the pouch for inflation. The web can be cut along cut line C to form separated inflatable products. As illustrated by FIG. 20C, the inflatable product 1000 can be positioned about an object 1020 with its fastening regions engaged.

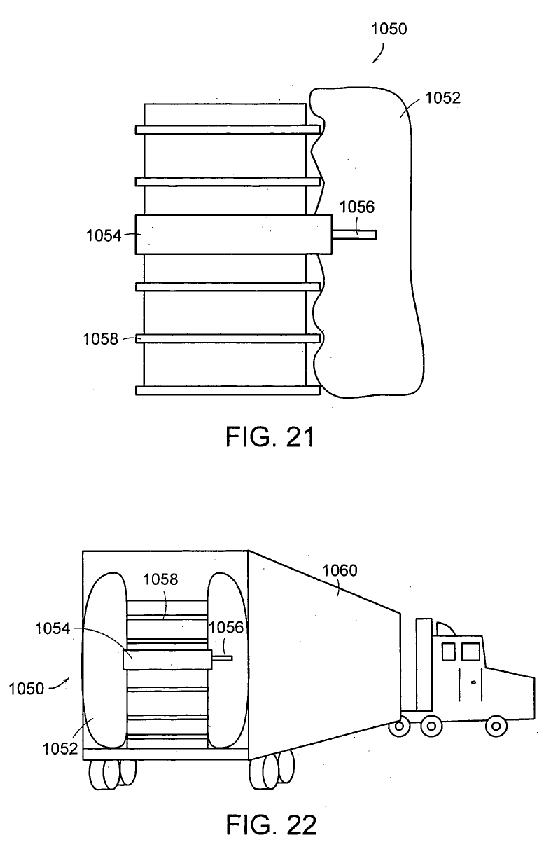

As mentioned above, the inflatable products can be sized and shaped as desired. Referring to FIGS. 21 and 22, an inflatable product 1050 having a relatively large inflatable pouch 1052 (e.g., from about 1 foot to about 5 feet wide and from about 1 foot to about 7 feet long), a strap 1054 extending from the pouch and a valve 1056 for inflating the pouch is used as a dunnage bag (e.g., for securing truck loads to avoid damage during shipping due to slippage and shifting). The strap 1054 is sized to extend about stacked crates 1058 and to engage matable regions of fastener elements for releasable securement.

Referring now to FIG. 22, a pair of the inflatable products 1050 is shown securing crates 1058 along the centerline of a trailer 1060. Other arrangements can also be used, such as side bracing and front-to-rear bracing.

A number of embodiments of the invention have been described. Nevertheless, it will be understood that various modifications may be made without departing from the spirit and scope of the invention.

What is claimed is:

1. An inflatable product comprising:
overlapped areas of flexible material defining therebetween an inflatable pouch, the pouch bounded by bonded regions of the flexible material and defining a limited opening for inflating the pouch,
wherein the flexible material includes multiple fastening regions including
a first fastening region in which an array of fastener elements with stems are arranged, the stems integrally molded with and extending from a common resin substrate, and
a second fastening region of fibrous loops releasably engageable with the fastener elements.

2. The inflatable product of claim 1 further comprising a valve to inhibit egress of fluid from the inflatable pouch through the limited opening, once inflated.

3. The inflatable product of claim 2, wherein the valve defines a passage that is collapsible in response to internal pouch pressure.

4. The inflatable product of claim 3, wherein the valve comprises two sheets of flexible material sealed together in face-to-face relation along either side of the passage.

5. The inflatable product of claim 2, wherein the valve comprises an inlet and a plug configured to seal the opening.

6. The inflatable product of claim 1, wherein the resin substrate from which the fastener element stems extend comprises a region of the flexible material.

7. The inflatable product of claim 1, wherein the second fastening region overlaps the pouch.

8. The inflatable product of claim 1, wherein the array of fastener element stems overlaps the pouch.

9. The inflatable product of claim 1, wherein the second fastening region covers substantially all of a front side of the product.

10. The inflatable product of claim 1, wherein the resin substrate is bonded to and extends from an edge of the flexible material.

11. The inflatable product of claim 1, wherein the pouch comprises multiple hydraulically connected compartments defined between extensions of the bonding regions.

12. The inflatable product of claim 1, wherein the opening is defined within a fitment secured to the flexible material.

13. The inflatable product of claim 12, wherein the fitment is configured to mate with a flexible hose for hydraulic communication with the pouch.

14. The inflatable product of claim 1 further comprising a compartment defined between adjacent areas of the flexible material and bounded by bonded regions of the flexible film material, the compartment defining an opening for receiving an object.

15. The inflatable product of claim 14, wherein the first and second fastening regions are positioned at the opening of the compartment to form a releasable closure for releasably closing the opening of the compartment.

16. The inflatable product of claim 14, wherein the compartment and the inflatable pouch share a wall of flexible material.

17. The inflatable product of claim 14 further comprising a thermal pad positioned within the compartment.

18. The inflatable product of claim 1, wherein the flexible material forms a strap carrying at least one of the first and second fastening regions, the strap being of sufficient length to wrap about an object and engage the first and second fastening regions to secure the pouch to an object.

19. The inflatable product of claim 1, wherein the flexible material comprises film.

20. The inflatable product of claim 19, wherein the film has a thickness of between about 0.0005 and 0.008 inch.

21. The inflatable product of claim 20, wherein the film has a thickness between about 0.001 and 0.005 inch.

22. The inflatable product of claim 21, wherein the film has a thickness of about 0.004 inch.

23. The inflatable product of claim 19, wherein the flexible material comprises polyethylene.

24. The inflatable product of claim 1, wherein the fibrous loops comprise fibers formed from a loop material selected from a group consisting of a non-woven material, a woven material and a knit material.

25. The inflatable product of claim 24, wherein the loop material is bonded to the flexible material.

26. The inflatable product of claim 24, wherein the loop material comprises a dimensionally stable loop material.

27. The inflatable product of claim 1, wherein the fastener elements include loop-engageable heads extending laterally from the stems in discrete directions.

28. The inflatable product of claim 1, wherein the fastener elements include loop-engageable heads extending laterally from the stems in multiple directions.

29. The inflatable product of claim 1, wherein the fastener elements and fibrous loops are configured to enable disengagement of the first and second fastening regions by application of a peeling force of less than about 0.2 pounds of force per transverse inch of engaged width of the engaged fastener elements.

30. The inflatable product of claim 29, wherein the fastener elements and fibrous loops are configured to enable disengagement of the first and second fastening regions by application of a peeling force of less than about 0.1 pounds of force per transverse inch of engaged width of the engaged fastener elements.

31. The inflatable product of claim 1 having an overall weight of less than about 150 grams per square meter of overall area of one broad side of the product in an uninflated state.

32. The inflatable product of claim 1 in the form of a blood pressure cuff.

33. The inflatable product of claim 32 comprising a fitment for attachment to a flexible hose, the fitment providing access to the pouch.

34. The inflatable product of claim 33 comprising a second fitment.

35. The inflatable product of claim 33, wherein the fitment defines more than one passages.

36. The inflatable product of claim 1 in the form of a reclosable bag, the flexible material defining a compartment for containing an object.

37. The inflatable product of claim 1 in the form of a splint for confining a body part of a patient.

38. The inflatable product of claim 37, wherein the splint defines a hole therethrough, shaped to receive a part of a human body.

39. The inflatable product of claim 1 in the form of a floatation device and configured to be strapped about a human body.

40. The inflatable product of claim 39 in the form of a life jacket, wherein the pouch capable of inflating to an amount capable of keeping a person afloat in water.

41. An inflatable blood pressure cuff comprising:
a compartment defined between overlapped areas of flexible film material and bound by bonded regions of flexible film material, the compartment defining an opening sized to receive an inflatable pad; and
an inflatable pad positioned within the compartment, the compartment configured to allow the inflatable pad to expand while positioned within the compartment,
wherein the flexible film material includes multiple fastening regions including a first fastening region in which an array of fastener elements including stems are arranged, the stems integrally molded with and extending from a resin base, and a second fastening region of fibrous loops releasably engageable with the fastener elements.

42. The inflatable blood pressure cuff of claim 41, wherein the inflatable pad comprises a fitment allowing communication with the inflatable pad.

43. The inflatable blood pressure cuff of claim 41, wherein the fitment is configured to mate with a flexible hose for hydraulic communication with the pad.

44. The inflatable blood pressure cuff of claim 41 wherein the second fastening region overlaps the compartment.

45. The inflatable product of claim 41, wherein the fibrous loops of the second fastening region comprise a dimensionally stable loop material that inhibits an increase in bounded area of the compartment during inflation.

46. A method of forming an inflatable product, the method comprising:
overlapping two areas of flexible sheet material;
bonding the overlapped areas in selected regions to form a pouch defined between overlapped areas and bounded by the bonded regions, the pouch defining a limited opening for inflating the pouch; and
including on the sheet material both an array of fastener elements comprising stems integrally molded with and extending from a common resin substrate, and a region of fibrous loops carried by the sheet material and releaseably engageable with the fastener elements.

47. The method of claim 46 further comprising attaching a valve to the pouch at the limited opening.

48. The method of claim 47, wherein the valve defines a passage that is collapsible in response to internal pouch pressure.

49. The method of claim 47, wherein the valve comprises two sheets of flexible material sealed together in face-to-face relation along either side of the passage.

50. The method of claim 47, wherein the valve comprises an inlet and a plug configured to seal the opening.

51. The method of claim 46, wherein the array of fastener elements are formed by introducing a molten resin to a gap defined adjacent a mold roll surface, the mold roll including an array of cavities for molding fastener element stems, the resin introduced such that resin enters the cavities to form fastener element stems while excess resin forms the substrate.

52. The method of claim 51 comprising forming the sheet material of the molten resin.

53. The method of claim 51 comprising introducing a preformed sheet material to the gap under conditions to bond the substrate to a surface of the sheet material.

54. The method of claim 51 comprising post-forming distal ends of the stems to form engageable heads extending outwardly from the stems.

55. The method of claim 51, wherein the cavities are shaped to form loop-engageable fastener elements.

56. The method of claim 51 comprising introducing a preformed loop material to the gap under conditions to permanently bond the loop material to the sheet material.

57. The method of claim 46 comprising:
overlapping two different areas of the flexible material; and
bonding the different overlapped areas in selected regions to form a second pouch defined between overlapped areas and bounded by the bonded regions.

58. A method of measuring blood pressure, the method comprising: providing an unused blood pressure cuff comprising
overlapped areas of flexible material defining therebetween an inflatable pouch, the pouch bounded by bonded regions of the flexible material and defining a limited opening for inflating the pouch;
wherein the flexible material includes multiple fastening regions including
a first fastening region in which an array of fastener elements with stems are arranged, the stems integrally molded with and extending from a common resin substrate; and
a second fastening region of fibrous loops releasably engageable with the fastener elements;
wrapping the blood pressure cuff around a portion of a patient's body;
inflating the pouch to a pressure associated with blood pressure measurement;
removing the blood pressure cuff from around the portion of the patient's body; and
discarding the blood pressure cuff.
